(19) United States
(12) Patent Application Publication
(10) Pub. No.: US 2023/0242963 A1
(43) Pub. Date: Aug. 3, 2023

(54) COMPOSITIONS FOR EXPOSING FILM-FORMING MICROBES AND METHODS FOR USE OF THE COMPOSITIONS

(71) Applicant: Guild Associates Inc., Dublin, OH (US)

(72) Inventor: Augustine Dinovo, Charleston, SC (US)

(21) Appl. No.: 18/163,862

(22) Filed: Feb. 2, 2023

Related U.S. Application Data
(60) Provisional application No. 63/306,057, filed on Feb. 2, 2022.

(51) Int. Cl. C12Q 1/24; C12Q 1/10; C12N 9/22; C12N 9/64; C12N 9/76; C12N 9/26; C12N 9/46

(52) U.S. Cl. CPC C12Q 1/24; C12Q 1/10; C12N 9/22; C12N 9/6491; C12Y 304/24007; C12N 9/6472; C12Y 304/22002; C12N 9/6427; C12Y 304/21004; C12Y 302/01001; C12N 9/2414; C12N 9/2454; C12Y 302/01011; C12Y 304/21062; C12N 9/6424

(57) ABSTRACT

Current methods for detection of microbial contaminants on surfaces use swabbing/wiping to extract microbes for analysis. This removes easily transferable microbes but fails to extract microbes living in biofilms, which reduces sensitivity and may mask the true degree of contamination. The current disclosure provides an enzyme cocktail that disrupts the biofilm and improves the extraction of live microbes for analysis. Applicant's enzyme system is particularly useful for the application to a variety of surfaces, but particularly on a variety of food processing surfaces. Utilization of Applicant's enzyme cocktail makes possible the extraction of a representative sample of live microorganisms present on a surface, including film forming microorganisms, without affecting non-film forming microorganisms also present on a surface.

### CROSS-REFERENCES TO RELATED APPLICATION

This application claims the benefit of U.S. Provisional Patent Application No. 63/306,057, filed Feb. 2, 2022, and entitled COMPOSITIONS FOR EXPOSING FILM-FORMING MICROBES AND METHODS FOR USE OF THE COMPOSITIONS which is incorporated herein by reference.

### GOVERNMENT LICENSE RIGHTS

This invention was made with government support under contracts 2021-33530-34362 and 2022-33610-37827 awarded by the U.S. Department of Agriculture. The government has certain rights to this invention.

### BACKGROUND

Food borne illnesses continue to be a substantial burden, and health threat across the globe. Estimates from World Health Organization for 2010 indicate that globally over 580 million food-borne illnesses from infectious agents occurred resulting in over 347,000 deaths. Estimates from the USA's Center for Disease Control for 2000 to 2008 indicate annually there were about 9.4 million food-borne illnesses, 55,961 resulting hospitalizations, and 1,351 deaths during that time period. Bacterial pathogens are key players and have produced the most hospitalizations and deaths compared to other pathogen groups. Examples of bacteria of concern in the food production industry include but are not limited to Campylobacter jejuni, Salmonella enterica, Listeria monocytogenes, Escherichia coli, Pseudomonas fluorescens, Acinetobacter johnsonii, Lactobacillus plantarum, and Serriatia proteamaculans. However, other microbes such as fungus can also produce biofilms. Some examples of biofilm producing fungi are Candida spp., Aspergillus spp., Cryptococcus neoformans, Fusarium spp., Blastoschizomyces capitatus, Malassezia pachydermatis, Pneumocystis spp., Trichosporon asahii, Rhizopus spp., and Rhizomucor spp.

As a result, surfaces involved in food processing and medical facilities are regularly monitored for possible contamination. Microorganisms present on surfaces can form colonies having a variety of forms including biofilms. Biofilms are complex communities of microbes enmeshed in a web of extracellular polymeric substances which binds the community together and to a surface and provides protection from adverse conditions (including disinfection), removal, and prevents easy extraction for monitoring. The food industry has appreciated the risk of biofilms on food processing surfaces for some time, but despite the cleaning and disinfection procedures routinely applied, there is substantial evidence that these procedures don't fully remove bacteria within biofilms. This failure can allow pathogenic and food spoilage bacteria to be transferred to food products during processing leading to food borne illnesses. The “resident” bacteria (bacteria repeatedly isolated from the same food processing surfaces despite cleaning and disinfection) are generally nonpathogenic, but these and food pathogens can construct or invade biofilms on food processing surfaces making removal and/or destruction more difficult.

Currently, the food industry monitors bacterial contamination in their facilities and on their equipment. Traditional culture-based methods are giving way to PCR, antibody-based systems, and even sequencing based methods can provide pathogen specific information much more quickly. Additionally, very rapid highly sensitive nonspecific assays that quantify adenosine triphosphate (ATP) from biological sources have been developed to monitor general hygiene on surfaces.

Despite these advanced detection tools, the current surface sampling techniques do not efficiently extract microbes in biofilms reducing assay sensitivity and masking true contamination risk. Applicant's disclosure describes the development of an enzymatic cocktail that dissolves the biofilm on the surfaces of manufactured equipment and structures while maintaining the viability of representative microbes therein (and other microbes present outside of the biofilm) so they can be easily extracted from surfaces for monitoring, increasing the sensitivity, accuracy, and reliability of essentially all microbial detection technologies.

### SUMMARY

Monitoring the microbial status of food contact surfaces is a requirement in the food industry. The traditional method has been culture-based testing where the surface is sampled either by swabbing or contacting it with an agar plate or slide which is then cultured to detect total aerobic bacterial counts or specific pathogens. The culture can take several days and the method will not detect microbes that don't grow on the particular culture plate media or selected growth conditions (i.e., nonaerobic), which is not ideal. Newer methods use PCR (polymerase chain reaction) which if used without culture can be relatively quick, more sensitive, and can be multiplexed to identify multiple pathogens specifically and simultaneously. In addition, next generation sequencing methods can provide more comprehensive pathogen information and if using meta-analysis can even provide community level information. Currently these later test methods are too costly for routine analysis of food processing surfaces.

These noted test methods provide pathogen identification, but the total microbial load on surfaces is also of interest. Total plate counts historically are used, but they are biased towards aerobic easily culturable microbes, are laborious, and slow. The most common current method is the ATP luminescence assay which measures total ATP on surfaces. All living things produce ATP making its measurement useful for measuring the abundance of microbial life. The ATP assay uses firefly luciferase which converts ATP to ADP producing light (measured in relative light units (RLU)) which is proportional to the ATP present. The assay solution also has reagents that lyse bacterial cells releasing ATP for the luciferase. The assay is highly sensitive and commercial products developed for this application provides sample-to-answer in minutes.

However, biologically derived fluids such as blood, meat-processing juices, particulates, and even exfoliated human skin (biological soils) can contain ATP confusing assay interpretation. Residual disinfectants and cleaning agents can also influence ATP signal. Nevertheless, the ATP test is widely utilized in the food industry as a measure of general hygiene because surfaces properly cleaned should not have either microbes or biological soils present ideally.

Biofilms are ubiquitous in nature. It is now commonly believed that most microbial life is found in biofilms. The gel-like qualities of the biofilm's extracellular polymeric substances provide protection from harsh environments and chemical treatments (including detergents disinfectants, biocides, and antimicrobials), and resists extraction of materials from film located on surfaces. It achieves this by preventing diffusion of lethal compounds into its interior while strong surface attachment resists mechanical removal, and its cross-linked nature foils chemical removal. All contamination testing systems discussed above use swabbing or contact plates to sample surfaces which can collect microbes weakly associated with surfaces but can't efficiently extract microbes embedded in biofilms. After cleaning and disinfection, the upper and/or more vulnerable surface layers may have been removed leaving the deep tightly enmeshed microbes on the surface. The cleaning and disinfection treatment initially may do no more than reduce the population of the biofilm. Swabbing such hardened residual communities will be even less efficient at collecting bacteria than swabbing a virgin biofilm because biofilms are well known to recover from such conditions. The inability of current monitoring systems to detect pathogens deeply embedded in biofilms masks the true surface contamination and risks promoting food borne illnesses rather than preventing them, by providing false negative results.

Clearly the food industry needs new surface sampling technology that can efficiently extract bacteria from biofilms on food processing/handling surfaces increasing the sensitivity, accuracy, and reliability of surface contamination monitoring systems and reduce food-borne illnesses.

In addition to the food industry (farm to table), there are many other applications where microbes commonly generate biofilms. These biofilms cause problems in these applications (examples: biofouling, biocorrosion, and illness) and the invention describe should be advantageous to these applications. Some, but not all of these applications include hospitals, chemical pipelines, home or businesses, restaurants, animals (mammals, humans, etc.), production processes, water distribution and cleaning systems, HVAC, equipment, farms, milk production, groceries, medical devices, and those doing research on biofilms. Microorganisms known to form biofilms within medical facilities include, but are not limited to Enterococcus faecium, Staphylococcus aureus, Klebsiella pneumoniae, Acinetobacter baumannii, Pseudomonas aeruginosa, Enterobacter spp, E. coli, and Candida spp.

Another application of the innovation includes using the enzyme complex in combination with therapeutic substances. The enzyme complex would break down the biofilms to allow access for the therapeutic substance to the microbes that create the biofilm. This access will allow the therapeutic substances to perform their therapeutic effect directly on the microbes that had been hiding in the biofilm and reduce the chances of reestablishment of the microbe.

A first aspect of the current disclosure includes a method for disrupting a film formed by a colony of film-forming microbes present on a surface of a manufactured article. The method can be utilized before or after a cleaning process and involves contacting the film with an enzyme cocktail for a time sufficient to disrupt the film and expose and or release viable microbes for sampling. The film forming microbes can include bacterial, fungi, and combinations thereof. One embodiment of the enzyme cocktail includes an enzyme selected from the group consisting of DNase, collagenase, combinations thereof and at least one additional enzyme. The at least on additional enzyme can include an enzyme selected from the group consisting of proteases, polysaccharides, and combinations thereof. Additionally, for some embodiments, the at least one additional enzyme can include alpha-amylase, Beta acetylhexosaminidase, polygalacturonase, Dextranase, Mutanase, Cellulase, Trypsin, Papain, Glutamyl endopeptidase, Actinidin, Proteinase K, and Savinase.

A further aspect of the method disclosed includes the further step of removing viable microbes from the surface of a manufactured article for testing after contacting the film with the enzyme cocktail. The further testing can involve identifying the presence or absence of a specific microbe. Testing methods that have proven particularly useful include, but are not limited to ATP luminescence, PCR, DNA sequencing, and aerobic plate count (APC), crystal violet assay, and BTG assay. Microbes typically encountered in testing include, but are not limited to include Campylobacter jejuni, Salmonella enterica, Listeria monocytogenes, Escherichia coli, Pseudomonas fluorescens, Acinetobacter johnsonii, Lactobacillus plantarum, and Serriatia proteamaculans.

A further aspect of the current disclosure involves an enzyme cocktail that includes water and DNase, collagenase, and combinations thereof and at least one additional enzyme. The enzyme cocktail is configured for applying to a microbial film formed by one or more film-forming microbes positioned on a surface of a manufactured article to release viable microbes from the film. The enzyme cocktail is configured for application to the surface of the manufactured article having any orientation in space. The manufactured article includes articles made by hand or machinery and is distinct from structural body parts having a surface and produced by members of the animal kingdom. Examples of manufactured articles include, but are not limited to, walls, floors, tables, equipment, drains, counters, machines, containers, and the like.

The at least one additional enzyme is selected from the group of enzymes including alpha-amylase, Beta acetylhexosaminidase, polygalacturonase, Dextranase, Mutanase, Cellulase, Trypsin, Papain, Glutamyl endopeptidase, Actinidin, Proteinase K, and Salvinase. Certain embodiments include an enzyme cocktail having at least two additional enzymes from the above selection of enzymes. In order to provide consistent coverage for surfaces having all configurations and reduce the rate of water evaporation after the application to a surface, certain embodiments of the enzyme cocktail can include an emulsifying agent and exist as an emulsion.

A still further aspect of the current disclosure includes the surface of a manufactured article that includes a biofilm thereon that includes at least one film-forming microbe where the film has been degraded by contact with an enzyme cocktail to release viable microbes available for sampling where the enzyme cocktail includes water, DNase, collagenase, and combinations thereof and at least one additional enzyme. Certain embodiments of the enzyme cocktail include an emulsifying agent to cause the enzyme cocktail to form an emulsion. Surfaces found on the manufactured articles include, but are not limited to plastic or polymeric surfaces, metal surfaces (typically stainless-steel surfaces), wooden surfaces, coated surfaces, and the like.

A still further aspect of the current disclosure involves treatments of bacterial films with embodiments of the disclosed enzyme cocktail that do not result in appreciable loss of bacterial viability. However, this does not prevent the enzyme cocktail's combination with additional agents that are lethal to bacteria resulting in the enzyme-exposed bacteria to being killed. The additional agents can include, but are not limited to, antibiotics, bacteriostatic agents, antifungals, and the like.

A still further aspect of the current disclosure includes a kit including one of the enzyme cocktails described above, an applicator for applying the enzyme cocktail to a film produced by one or more film-forming microbes on a surface of a manufactured article, and a tool configured for removing released and viable one or more film-forming microbes. Suitable applicators for applying the enzyme cocktail include, but are not limited to, a sprayer, a brush, a roller, a dropper, a sponge, a wipe, a swab, and a spreader. An appropriate applicator could also be prewetted with the enzyme cocktail. Suitable tools for removing the released and viable microbes include, but are not limited to, a knife, a scraper/pick, a swab, a wipe, a sponge, and a vacuum device. Any specific kit can contain any combination of tools for removing microbes from the disrupted film.

### DESCRIPTION

The extracellular polymeric substances of biofilms are composed primarily of polysaccharides, proteins, and extracellular DNA (eDNA). Certain enzymes have been found to break down individual components of extracellular polymeric substances, and enzyme cocktails have been formulated with various cleaning reagents (e.g. detergents, dispersing agents, wetting agents etc.) to produce enzymatic cleaners for cleaning surfaces with efficacy competitive with top chemical surface cleaners. However, such products are designed for killing microbes and are unsuitable for non-lethal extraction of microbes.

Enzymes having beneficial activity for inclusion in enzyme cocktails designed for the destruction of biofilms were determined by screening for biofilm disrupting activity using relevant bacteria that form biofilms on the surface of relevant manufactured articles and selecting the candidate combination that was the most effective against the biofilms produced by the greatest number of bacteria. Specific to this disclosure maintaining microbe viability, and the ability to collect microbes released by biofilm-disrupting effects from manufactured surfaces are also important aspects of the disclosure. The approach used in this disclosure considers that extracellular polymeric substances in biofilms can be composite fibers from a combination of microbes and that combining enzymes that attack distinct components of the biofilm may functionally synergize dissolution to break down fibers in the biofilm more effectively than mono-enzyme treatments.

Commercial anti-biofilm enzymes are not designed for maintaining microbe viability which is necessary for selecting suitable enzyme cocktails to destroy the biofilm without reducing the surface's functioning bacterial population and provide an accurate measurement of the surface's bacterial population. As a result, only enzymes that can destroy the biofilm's extracellular structure on manufactured surfaces while maintaining the viability of the released bacteria should be included in the enzyme cocktail which is the focus of this disclosure. For testing purposes, a specific group of biofilm forming bacteria were selected and a group of potential enzymes were similarly selected.

### The Selection of Bacteria for Testing:

Thorough consideration of the bacteria selected for testing was important for the test design because the specific structural components of their biofilms can differ based on the bacterial system producing the biofilm. The utilization of an insufficient species breadth in the study would limit an enzyme cocktail's effectiveness, yet too large a panel risks would become excessively laborious and costly without gaining proportional value. In the case of food processing surfaces, consideration of non-pathogenic bacteria that typically form biofilms on such surfaces are important because they are known to resist cleaning and disinfection treatments and can potentially harbor and protect pathogens within their biofilms.

Dozens of microorganisms may be present in any food processing environment forming complex and poorly understood communities of microorganisms. In order to simulate this pattern in the test system, four (4) bacteria representing a resident community were included in the testing along with three (3) food borne pathogens (Table 1). Certain species were selected for their ability to cause food-borne illnesses. Other species were selected because they were commonly found to produce biofilms on surfaces of manufactured articles utilized in the food industry, but are not strictly considered pathogens (termed resident bacteria). The three (3) pathogens selected are dominant causes of food borne illnesses, are regularly isolated from food processing work surfaces, and are easily cultured using common culture practices. The bacteria selected were also generally guided by a focus on species associated with surfaces in contact with meat and poultry commodities because these are a dominant source of food borne illnesses. Table 1 provides a listing of the bacteria utilized in the applicant's disclosure research.

TABLE 1
Bacterial Species Utilized

| Foodborne Pathogens | Resident Bacteria |
|---|---|
| Escherichia coli O157:H7 | Pseudomonas fluorescens |
| Salmonella enterica | Acinetobacter johnsonii |
| Listeria monocytogenes | Lactobacillus plantarum |
| | Serratia proteamaculans |

### Selection of Enzymes for Testing:

For this initial study, well established commercial enzymes are used for testing. Table 2 lists the selected enzymes and some characteristics associated with each of them.

TABLE 2
Candidate enzymes and their characteristics

| Preliminary Enzymes | Enzyme Type | Enzyme Subtype/Activity |
|---|---|---|
| DNase I | Nuclease | DNA degrading |
| alpha-amylase | Polysaccharidase | α-1,4 endoglycocidase |
| β-N- | Polysaccharidase | β-N- |
| acetylglucosaminidase | | acetylglucosaminidase |
| Polygalacturonase | Polysaccharidase | poly-alpha-1,4-galacturonide |
| (PG) | | glycanohydrolase |
| Dextranase | Polysaccharidase | β-1,6 glucanase |
| Cellulase | Polysaccharidase | β-1,4 glucanase |
| Trypsin | Protease (serine) | Cleaves at basic aa |
| Papain (from papaya) | Protease | Cleaves at basic amino acids |
| | (cysteine) | preceded by a hydrophobic aa |
| Glutamyl | Protease (serine) | Cleaves at acidic aa |
| endopeptidase | | |
| Collagenase | Protease | Collagen cleavage |
| | (cysteine) | |
| Proteinase K | Protease (serine) | Cleaves at hydrophobic aa |
| Savinase | Protease (serine) | A subtilisin, nonspecific |
| | | cleavage |

The selected enzymes represent three (3) enzyme classes: proteases, polysaccharidases, and nucleases. The selection focused on producing a diverse set of enzymatic activities within each of these main classes of enzymes because the bacteria selected encompass several genera and can have diverse biofilm compositions.

The disclosed research involves the testing of a set of commercially available (low cost) enzymes (listed in Table 2) against a panel of food-borne pathogens and resident bacteria (listed in Table 1) to identify an enzyme cocktail capable of disintegrating food industry relevant biofilms and increasing bacterial release/extraction from populated surfaces without substantial loss of viability. The enzyme cocktail developed is expected to be applicable for all current surface contamination monitoring systems used by the food industry, especially culture-based systems. Non-culture-based PCR or DNA sequencing systems will benefit as well because directly extracting DNA from surfaces may leave appreciable amounts of DNA bound to a surface, while extracting cells whole and then lysing and extracting the DNA in a testing tube is expected to result in higher recovery. If used with the ATP assay, ATP testing with and without Applicant's enzyme cocktail allows for specific detection of microbes associated with biofilms through the differential ATP levels providing a capability it currently lacks. Thus, the utilization of Applicant's enzyme cocktail is valuable to food processing safety managers in the food industry through its ability to allow contamination monitoring systems to detect and identify microbes on surfaces with higher sensitivity, accuracy, and reliability. This allows food processing surfaces to be better sanitized and reduce the incidence of food borne illnesses in the country and the world.

### Applicant's Test Strategy:

Applicant's initial testing was carried out with combinations of two (2) enzymes rather than a single enzyme with a nuclease present in all enzyme combinations. DNase I, a specific commonly utilized DNA nuclease, was selected as a universal enzyme. Both DNase I and other DNA nucleases were compared and formulated to have equivalent activity and then used interchangeably in the research. Nuclease was tested against all seven (7) species at three (3) concentrations and then the most overall effective concentration was used in two (2) enzyme testing. Initial two (2) enzyme testing utilized traditional mono-species biofilms of the 7 species in Table 1. The nuclease was also always tested alone in all experiments allowing some determination of its contribution to the resulting antibiofilm activity. Non-nuclease enzymes were tested at 3 concentrations while the nuclease concentration was held constant. Anti-biofilm activity was measured using the Crystal Violet assay, an easy and ubiquitous assay used for measuring changes in biofilm magnitude in the biofilm research industry.
Two (2) enzyme combinations that showed especially high activity by the Crystal Violet were further evaluated. These enzyme combinations were tested for bacterial cell release from enzyme-treated biofilms. Post-enzyme exposure sample overlying solutions were collected and centrifuged to collect bacterial cells released by the enzyme treatment. The supernatants were removed, and the cell pellet resuspended in PBS and ATP luminescence was determined and compared to non-enzyme treated controls. Preferred enzyme pairs were expected to release more cells than untreated controls. Additionally, because maintaining extracted microbe viability was important, the ATP luminescence of the post-centrifugation cell free supernatants were also determined and compared to untreated controls. ATP is normally sequestered inside of cells so the presence of substantially more ATP in the cell free supernatant of enzyme treated biofilms indicates the enzyme treatment may have had toxic effects on the treated bacteria. Testing using a model quaternary ammonium substance (QAS) sanitizing agent at functionally active concentrations resulted in over twelve-fold higher cell-free ATP luminescence than untreated control biofilms in a representative species demonstrating the ability of the test to detect toxic effects on the bacteria.

Additionally, multi-species biofilms were composed and tested using the bacteria listed in Table 1. It is well understood that in nature multi-species (MS) biofilms are the typical condition encountered. The complexity of such systems and our inability to understand and model these complex systems has limited most biofilm studies to mono-culture models. In vitro studies using two (2) or more species biofilms are more common in food safety research as it is appreciated that these complex systems are ubiquitous in the food processing environment, and dual species biofilms indicate that such biofilms can generally protect microbes from disinfectants and other stresses better than single-species biofilms. Applicant's enzyme cocktails (enzyme cocktail) developed using mono-culture biofilms face a greater challenge disrupting the more rugged multi-species (MS) biofilms.

Multi-species biofilms were screened by treatment with a sanitizing agent to identify species combinations that show heightened resistance. The multi-species biofilm with the most resistance to the sanitizer was used to screen three (3) enzyme combinations for anti-biofilm and bacterial release testing of the six (6) top performing two (2) enzyme combinations tested earlier. Non-nuclease enzymes were tested at two (2) different concentrations. Each three-enzyme combination was also compared to each of the two-enzyme combination core pair to try to identify synergy of the three-enzyme combination over the two-enzyme combination.

In addition, S. proteamaculans as a single species biofilm had a Crystal Violet % Ctr average value across all enzymes tested of just 78% Ctr (i.e., only an average 22% biofilm reduction), showing it was especially resistant to enzyme treatment. Therefore, three-enzyme combinations were also tested against single species biofilms of this species.

The results of the initial two enzyme combination testing using the Crystal Violet assay is given in Table 3 and are given as a percent of the untreated control Crystal Violet signal.

TABLE 3
Biofilm Reduction Due to Enzyme Cocktail Treatment in Model Species

| Enzyme | | | | | | | | 7-species |
|---|---|---|---|---|---|---|---|---|
| Combined w/nuclease | E. coli | S. enterica | L. monocytogenes | S. proteamaculens | A. JohnsonII | P. fluorescens | L. plantarum | average |
| Proteinase K | 100% | 100% | 72% | 0% | 86% | 19% | 71% | 64% |
| Trypsin | 50% | 54% | 92% | | 60% | 29% | 56% | 57% |
| Papain | 100% | 28% | 100% | 14% | 60% | 42% | 27% | 53% |
| Savinase | 25% | 73% | 90% | 39% | 85% | 73% | 89% | 68% |
| Collagenase (relacing | 82% | 62% | 59% | 79% | 0% | 20% | 26% | 47% |
| Actinidin) | | | | | | | | |
| Glutamyl (Glu-C) | | 22% | | | 50% | | | 36% |
| endopeptidase | | | | | | | | |
| Alpha amylase | 70% | 70% | 0% | 46% | | 31% | | 44% |
| Dextranase | 0% | 18% | 28.6% | 38% | 52% | 69% | | 34% |
| Dispersion B, (Beta | | 0% | | 0% | 0% | | | 0% |
| acetylhexosaminidase | | | | | | | | |
| Polygalacturonase | | | 28.7 | | | | | |
| Cellulase | 0% | 10% | 0% | 0% | 22.4% | | 0% | 5% |

Table 4 shows the results of the more comprehensive testing of select two-enzyme combinations which includes testing cell release from enzyme treatment as well as treatment toxicity.

TABLE 4a
Cell Release and Toxicity Values for Select
Enzyme Cocktails and Model Bacteria

| | Cell | Treatment |
|---|---|---|
| Bacteria/EC | Release | Toxicity |
| A. johnsonii, Proteinase K + Nuclease | 448.1% | 300.3 |
| L. monocytogenes Papain + Nuclease | 243.5% | 74.9% |
| L. monocytogenes Trypsin + Nuclease | 293.7% | 83.3% |
| L. monocytogenes Savinase + Nuclease | 65.8% | 106.1% |
| E. coli + Papain + Nuclease | 173.5% | 22.1% |
| E. coli + Proteinase K + Nuclease | 121.4% | 21.4% |
| E. coli + Collagenase + Nuclease | 114.4% | 40.0% |
| S. enterica, + alpha amylase + Nuclease | 391.6% | 62.1% |
| A. johnsonii, + Savinase + Nuclease | 222.1% | 57.4% |
| A. johnsonii, + Dextranase + Nuclease | 402.6% | 58.9% |
| S. proteamaculans + Dextranase + Nuclease | 78.5% | 37.1% |
| L. plantarum + Savinase + Nuclease | 191.4% | 18.4% |
| E. coli + alpha amylase + Nuclease | 92.6% | 66.9% |
| E. coli + Collagenase + Nuclease | 126.2% | 65.1% |
| S. proteamaculans + Collagenase + Nuclease | 202.0% | 56.7% |
| S. proteamaculans + Trypsin + Nuclease | 61.9% | 86.6% |

Cell release: Post-enzyme exposure sample overlying solutions were collected and centrifuged to pellet bacterial cells released by the enzyme treatment. The post-centrifugation supernatants were subsampled and removed, and the cell pellet resuspended in PBS and ATP luminescence was determined using the BacTiterGlo Microbial Cell Viability Assay (Promega) and compared to non-enzyme treated controls to give a percent of control value shown. Values over 100% were considered positively.
Treatment Toxicity: To measure cell toxicity the ATP luminescence of the post-centrifugation cell free supernatants were also determined using the BacTiterGlo Microbial Cell Viability Assay (Promega) and compared to untreated controls to give a percent control value shown. Values over 100% are viewed negatively as potential toxic effects of an enzyme treatment.

TABLE 4b
Validation of Toxicity Assay Using Known
Lytic Product and Representative Bacteria

| | Treatment |
|---|---|
| | Toxicity |
| Treatment | % Ctr |
| Biofilm Lifestyle | |
| s. proteamaculans + 10 μg/ml BZAC | 150.8% |
| s. proteamaculans + 100 μg/ml BZAC | 793.3% |
| s. proteamaculans + 1 μg/ml BZAC | 1250.5% |
| Planktonic lifestyle | |
| s. proteamaculans + 10 μg/ml BZAC | 284.9% |
| s. proteamaculans + 100 μg/ml BZAC | 724.9% |
| s. proteamaculans + 1 μg/ml BZAC | 859.3% |

Table 4b shows application of the ATP luminescence Treatment Toxicity Assay applied to a model QAS sanitizer, Benzalkonium chloride, at three (3) different concentrations grown in either biofilm or planktonic lifestyle using S. proteamaculans as a model organism. In multi-species biofilm testing, single species biofilm controls with this species consistently show biofilm reduction by treatment with 100 ug/ml BZAC making it a good species for testing BZAC toxicity. With both growth lifestyles a clear dose response curve is evident.

### Three Enzyme Combinations.

TABLE 5
Top 5 Three-enzyme combinations in either multi-species, or
S. proteamaculans biofilms (CVA-Crystal Violet Assay)

| | CVA | Cell Release |
|---|---|---|
| Three enzyme combination treatment | % Ctr | % Ctr |
| MS-biofilms | | |
| Collagenase + Papain + Nuclease, best | 4.50% | 213.80% |
| enzyme conc. | | |
| Collagenase + Papain + Nuclease, 2 | 4.60% | 215.00% |
| concentration ave | | |
| Trypsin + Papain + Nuclease, best enzyme | 5.10% | 481.80% |
| conc. | | |
| Trypsin + Papain + Nuclease, 2 | 5.70% | 543.80% |
| concentration ave | | |
| Collagenase + alpha-Amylase + Nuclease, best | 5.30% | 149.30% |
| enzyme conc. | | |
| Collagenase + alpha-Amylase + Nuclease, 2 | 5.80% | 198.50% |
| concentration ave | | |
| Collagenase + Dextranase + Nuclease, best | 5.70% | 254.50% |
| enzyme conc. | | |
| Collagenase + Dextranase + Nuclease, 2 | 6.90% | 281.40% |
| concentration ave | | |
| Trypsin + Dextranase + Nuclease, best | 7.20% | 507.10% |
| enzyme conc. | | |
| Trypsin + Dextranase + Nuclease, 2 | 8.30% | 544.40% |
| concentration ave | | |
| S. proteamaculans Biofilms | | |
| Collagenase + alpha-Amylase + Nuclease, | 29.10% | 89.20% |
| best enzyme conc. | | |
| Collagenase + alpha-Amylase + Nuclease, | 37.20% | 162.50% |
| 2 concentration ave | | |
| Collagenase + Savinase + Nuclease, best | 31.80% | 120.70% |
| enzyme conc. | | |
| Collagenase + Savinase + Nuclease, 2 | 49.60% | 94.70% |
| concentration ave | | |
| Collagenase + Dextranase + Nuclease, | 45.90% | 205.70% |
| best enzyme conc. | | |
| Collagenase + Dextranase + Nuclease, 2 | 54.60% | 228.90% |
| concentration ave | | |
| Collagenase + Papain + Nuclease, best | 56.80% | 150.80% |
| enzyme conc. | | |
| Collagenase + Papain + Nuclease, 2 | 61.80% | 150.70% |
| concentration ave | | |
| Savinase + Trypsin + Nuclease, best | 77.60% | 56.90% |
| enzyme conc. | | |
| Savinase + Trypsin + Nuclease, 2 | 85.40% | 58.70% |
| concentration ave | | |

Table 5 illustrates the top five (5) three-enzyme combinations for either the multi-species biofilms, or S. proteamaculans biofilms. Surprisingly, multi-species biofilms proved to be more vulnerable to enzyme combinations than S. proteamaculans biofilms. Nevertheless, multiple 3 enzyme combinations showed high activity against this resistant species. It is also noteworthy that collagenase is one component of 7 of the 10 three (3) enzyme combinations given in Table 5 making it an important member of the enzyme cocktail developed.

While applicant's disclosure has been summarized with reference to specific embodiments above, it will be understood that modifications and alterations in the embodiments disclosed may be made by those practiced in the art without departing from the spirit and scope of the invention. All such modifications and alterations are intended to be covered.

1. A method for disrupting a film formed by a colony of film-forming microbes on a surface of a manufactured article, the method comprising contacting the film with an enzyme cocktail for a time sufficient to disrupt the film and expose viable microbes for sampling.

2. The method of claim 1, where the enzyme cocktail includes an enzyme selected from the group consisting of dNase, collagenase, combinations thereof and at least one additional enzyme.

3. The method of claim 1, wherein the at least one additional enzyme includes an enzyme selected from the group consisting of proteases, polysaccharidases, and combinations thereof.

4. The method of claim 3, wherein the at least one additional enzyme is selected from the group consisting of alpha-amylase, Beta acetylhexosaminidase, polygalacturonase, Dextranase, Mutanase, Cellulase, Trypsin, Papain, Glutamyl endopeptidase, Actinidin, Proteinase K, and Salvinase.

5. The method of claim 1, further including removing viable microbes from the surface of a manufactured article for testing after contacting the film with the enzyme cocktail.

6. The method of claim 5, wherein the testing involves identifying the presence or absence of a specific microbe.

7. The method of claim 5, wherein the testing includes testing for a bacteria.

8. The method of claim 5, wherein the testing involves a test method selected from the group consisting of ATP luminescence, PCR, DNA sequencing, and aerobic plate count (APC), crystal violet assay, and BTG assay.

9. The method of claim 5, wherein the testing involves testing for bacteria selected from the group consisting of Campylobacter jejuni, Salmonella enterica, Listeria monocytogenes, Escherichia coli, Pseudomonas fluorescens, Acinetobacter johnsonii, Lactobacillus plantarum, and Serriatia proteamaculans.

10. The method of claim 5, wherein the testing involves testing for a fungus.

11. An enzyme cocktail comprising of water and at least one enzyme selected from the group consisting of dNase, collagenase, and combinations thereof and at least one additional enzyme,
wherein the enzyme cocktail is configured for applying to a microbial film formed by one or more film-forming microbes on a surface of a manufactured article to release viable microbes from the film, and
wherein the enzyme cocktail is configured for application to the surface of the manufactured article having any orientation.

12. The enzyme cocktail of claim 11, wherein the at least one additional enzyme is selected from the group consisting of alpha-amylase, Beta acetylhexosaminidase, polygalacturonase, Dextranase, Mutanase, Cellulase, Trypsin, Papain, Glutamyl endopeptidase, Actinidin, Proteinase K, and Salvinase.

13. The enzyme cocktail of claim 11 including at least two additional enzymes.

14. The enzyme cocktail of claim 13, wherein the at least two additional enzymes are selected from the group consisting of alpha-amylase, Beta acetylhexosaminidase, polygalacturonase, Dextranase, Mutanase, Cellulase, Trypsin, Papain, Glutamyl endopeptidase, Actinidin, Proteinase K, and Salvinase.

15. The enzyme cocktail of claim 11, further including an emulsifier configured to emulsify the enzyme cocktail.

16. The enzyme cocktail of claim 11, further including an agent to retard the evaporation of water.

17. The enzyme cocktail of claim 15, including at least one additional enzyme selected from the group consisting of alpha-amylase, Beta acetylhexosaminidase, polygalacturonase, Dextranase, Mutanase, Cellulase, Trypsin, Papain, Glutamyl endopeptidase, Actinidin, Proteinase K, and Salvinase.

18. A surface of a manufactured article including a biofilm thereon including at least one film-forming microbe, wherein the film has been degraded by contact with the enzyme cocktail of claim 11 exposing at least one viable microbe.

19. The surface of the manufactured article of claim 18, comprising a surface selected from the group consisting of a plastic surface, a steel surface, and a coated surface.

20. A kit including an the enzyme cocktail of claim 11, an applicator for applying the enzyme cocktail to a film produced by one or more film-forming microbes on a surface of a manufactured article, and a tool configured for removing released and viable one or more film-forming microbes.
